SEQUENCE LISTING (5 sequences)

SEQ ID NO: 1 - protein, 4 aa, Artificial sequence
  tetrapeptide adjacent to the beta-secretase cleavage site of human APP
EFRH

SEQ ID NO: 2 - DNA, 4659 bp, Artificial sequence
  single chain antibody
tcgagctcgcccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcgttagaacgcggctacaattaatacataaccttatgtatcatacacatacgatttaggtgacactatagaataacatccactttgcctttctctccacaggtgtccactcccaggtccaactgcacctcggttctatcgattgaattccccggggatcctctagagtcgacctgcagaagcttggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgggaattaattcggcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgttaacagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattaa

SEQ ID NO: 3 - DNA, 5428 bp, Artificial sequence
  Fab fragment
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 4 - DNA, 63 bp, Artificial sequence
  promoter/enhancer domain from the major immediate-early region of the human cytomegalovirus
ctctagaactagtggatcccccgggctgcaggaattcgcggccgcgaattcgatatcaagctt

SEQ ID NO: 5 - protein, 5 aa, Artificial sequence
  linker sequence
GGGGS

(19) United States
(12) Patent Application Publication
(10) Pub. No.: US 2011/0106062 A1
(43) Pub. Date: May 5, 2011

(54) System and Method For In Vivo Delivery of Antibodies and Fragments Thereof

(75) Inventors: Osiris Marroquin, Zurich (CH); Patrick Aebischer, Villette (CH); Maurizio Molinari, Bellinzona (CH); Nicolas Bouche, Les Pacots (CH)

(73) Assignee: ECOLE POLYTECHNIQUE FEDERALE DE LAUSANNE, Lausanne (CH)

(21) Appl. No.: 12/940,090

(22) Filed: Nov. 5, 2010

Related U.S. Application Data
(60) Provisional application No. 61/258,298, filed on Nov. 5, 2009.

(51) Int. Cl. A61M 37/00

(52) U.S. Cl. 604/890.1

(57) ABSTRACT

The present invention is directed to devices and methods for in situ delivering an antibody or a fragment thereof to a host. In particular, the invention relates to devices and methods for in situ delivering an antibody or a fragment thereof to patient suffering for a neurodegenerative disorder or other diseases treated by antibody administration.

### CROSS-REFERENCE TO RELATED APPLICATION

This application claims the benefit of U.S. Provisional Application Ser. No. 61/258,298, filed Nov. 5, 2009, the disclosure of which is hereby incorporated by reference in its entirety, including all figures, tables and amino acid or nucleic acid sequences.

### FIELD OF THE INVENTION

The present invention relates to controlled in vivo delivery of antibodies or fragments thereof using encapsulated cells for therapeutic purposes.

### BACKGROUND OF THE INVENTION

While advances in recombinant antibody technologies have allowed the production of antibodies to any antigen, engineered fragments of cloned antibodies have been designed to be produced in a various range of organisms such as E. coli, baculovirus, yeast and human cells. In particular, the recent development of humanized monoclonal antibodies has pushed interest and efforts towards the development of those molecules as therapeutics since they can specifically target disease specific factors while avoiding major secondary effects usually associated with conventional drug therapies. Further, engineered antibodies in the form of smaller recombinant antibody fragments (e.g. monovalent antibodies such as Fab or scFv) and engineered variants (diabodies, triabodies, minibodies and single-domain antibodies) are emerging as promising therapeutics (Hollinger et al., 2005, Nature Biotechnology, 23(9), 1126-1136; Devey et al., 2008, BioEssays, 30(9), 904-18).

However, if therapeutic monoclonal antibodies have undergone one of the fastest growing age in biopharmaceutical development, some concerns remain regarding their production, purification, administration and formulation. During production and administration, antibodies are susceptible to various modifications such as oxidation, proteolysis and aggregation which can alter their biological activities, induce side effects and/or limit their shelf life or administration regimen.

Further, the therapeutic use of antibodies via systemic delivery has encountered some major stumbling blocks, notably due to inflammatory responses (for example Fc-induced responses in the case of full antibodies), low activity due to the poor delivery to their site of action (for example in the case of CNS disorders) and/or to a short half-life and accumulation (for example in the case of antibody fragments such as single chain antibodies (scFv)). Those problems result in a limitation for a wide use of a large variety of antibodies due to cost and safety concerns.

Since delivery is a major issue for the therapeutic use of antibodies, the development of new delivery methods for antibodies that would lead to the use of lower doses, prolonged retention and slow clearance at the targeted site, while minimizing the immunological responses to those antibodies would be highly desirable.

### SUMMARY OF THE INVENTION

The present invention is based on the finding that the in vivo, in particular in situ, delivery of antibodies, in particular antibody fragments, through the use of device containing encapsulated cells capable of expressing, processing and secreting the said antibodies or fragments thereof is particularly advantageous. In particular, the invention is directed to novel methods and devices for the delivery of antibodies or fragments thereof directly to the Central Nervous System (CNS), for example for the treatment of neurodegenerative disorders, such as Alzheimer's disease.

According to a first aspect, the invention provides an implantable device for delivering an antibody or a fragment thereof to a host, comprising:

(a) A chamber comprising cells expressing and secreting said antibody or fragment thereof;

(b) A jacket encapsulating the chamber and providing a physical barrier between the said host and the chamber, the jacket having a permeability such that the secreted antibody or fragment thereof can diffuse therethrough into the host and the elements necessary for maintaining expressing cells alive can diffuse into the chamber.

According to a second aspect, the invention provides a method for delivering an antibody or a fragment thereof to a host, comprising implanting at least one device according to the invention into said host.

According to a third aspect, the invention provides a method for treating a mammal comprising delivering an antibody or a fragment thereof to a mammal through a device according to the invention.

According to a fourth aspect, the invention provides a use of a device according to the invention for the manufacture of a delivery system for delivering an antibody or a fragment thereof to a host.

### BRIEF DESCRIPTION OF THE FIGURES

FIG. 1 represents a device according to the invention used to encapsulate cells expressing and encoding an antibody, in particular a scFv such as the ones described in Example 3 (A) which is suitable for implantation in the brain parenchyma of mice such as described in Example 4 and its site of implantation (B). A: Examples of implantable devices according to the invention, configured as a hollow fiber without or with a refillable port ((1) or (2)), configured as a flat sheet without or with a refillable port ((3) or (4)) with the following features: a membrane configured to permits the passage of the secreted antibody or fragment thereof and to permits the passage of the elements necessary for maintaining expressing cells alive (5); a retrievable or fixed cap (6); a refilling tube (7); a membrane support (8); a chamber containing the secreting cells which may containing a preformed cell-supporting biocompatible matrix B: site of implantation of a device according to the invention in the mouse brain (a).

FIG. 2 represents anxiety-like behavior in APP23-scFvβ1 as determined in the L&D and EZM tests as compared to APP23, APP23-mock, and control aged matched WT-littermates as described in Example 4. A: Experiment design (M: months after APP23; (a): date of capsule implantation; (b): date of sacrifice); B and C: L&D test (D: distance in cm; T: time in seconds; (1): Light quadrant total; (2): Light quasdrant (w/o transition zone); (3): Transition zone); D and E: EZM anxiety-like test (D: distance in cm; T: time in seconds; (1): Total distance; (2): Close arms; (3): Open arms).

FIG. 3 represents scFvβ1 release and survival of encapsulated C2C12 cells as described in Examples 3 and 4. A: ELISA immunoassay measuring the amount of scFvβ1 (ng/24 hour) nreleased by C2C12-scFvβ1 capsules prior to implantation (a), and 6 months post-explantation (b); B: Hematoxylin-eosin staining performed on the retrieved capsules pre- (a) and post-implantation (b) showing the presence of numerous cells scattered within the PVA matrix (4×, scale bar 100 μm). C: Immunohistochemical detection of scFvβ1 using anti-histidine tag antibody in the right hemisphere of 6-month post-explanted capsules of APP23 mice. Left: APP23 mouse brain without surgery; Middle: APP23-mock mouse with bilateral implantation of capsules with control C2C12-mock cells; Right: APP23-scFvβ1 mouse with bilateral implantation of capsules with C2C12 cells expressing recombinant scFvβ1 antibody fragment (1×, scale bar 1 mm).

FIG. 4 represents the reduction of in vivo accumulation and production of Aβ in APP23-scFvβ1 (3) mice as as compared to APP23 (1) and APP23-mock mice (2). A: Congophilic stained sections of Aβ plaques from brains of APP23, APP23-mock and APP23-scFvβ1 mice (2×, scale bar 500 μm) B: Percentage area of positive congophilic staining (left: total Aβ; right: Hipoccampus and post-parietal Aβ); C: Amounts of Aβ₁₋₄₂ peptide (ng/mL) as determined by ELISA from brain protein extracts from soluble (left) and insoluble fractions (right) from posterior-parieta cortex (a) and hippocampus (b). Error bars: s.e.m. *p<0.05, **p<0.01 and ***p<0.001 determined by one-way ANOVA followed by Fischer least significant difference (LSD) post-hoc analysis.

FIG. 5 represents the plasmid maps for the mammalian expression plasmids used under Example 1 for expressing antibodies scFvβ1, IgGβ1 and Fabβ1. A: pRK5 vector used for inserting the antibody genes for scFvβ1 or IgGβ1. Features of the vector include a promoter/enhancer domain from the major immediate-early region of the human cytomegalovirus (CMV) of SEQ ID NO: 4; a multiple cloning region (MCS) including the following restriction sites listed from 5′ to 3′: XbaI, PstI, NotI, EcoRII, and HindIII for inserting the antibody gene of interest; a SV40 polyadenylation signals for RNA processing in mammalian cells; a SV40 origin for episomal plasmid amplification in COS cells; a bacteriophage f1 origin of replication for production of single-stranded plasmid DNA; an Ampicillin-resistant (AmpR) gene for amplification in E. coli bacterial strains; B: pcDNA3.1vector used for inserting the antibody gene for Fabβ1. Features of the vector include a promoter/enhancer domain from the region 232-819 of the human cytomegalovirus (CMV); a T7 promoter/priming site (863-882); a multiple cloning site (895-1010) for inserting the antibody gene of interest; a pcDNA3.1/BGH reverse priming site (1022-1039); a BGH polyadenylation sequence (1028-1252); a f1 origin (1298-1726); a SV40 early promoter and origin (1731-2074); a neomycin resistance gene (ORF) (2136-2930); a SV40 early polyadenylation signal (3104-3234); a pUC origin (3617-4287) (complementary strand); an Ampicillin resistance gene (bla) (4432-5428) (complementary strand); an ORF (4432-5292) (complementary strand); a ribosome binding site (5300-5304) (complementary strand); a bla promoter (P3) (5327-5333) (complementary strand).

### DETAILED DESCRIPTION OF THE INVENTION

The term “host” refers to an appropriate animal subject, including mammals and particularly human subjects in which the encapsulated cells that exhibit the desired cell property are implanted.

The term “cell” refers to a cell in any form, including but not limited to cells retained in tissue, cell clusters, and individually isolated cells. The cells in the present invention produce a biologically active molecule. The cells may be stem cells, primary cells, dividing cells or cell clones that naturally produce the biologically active molecule, or have been genetically engineered to do so. Typically, cells are fibroblasts or myofibroblasts or myoblasts, erythroblasts or ephitelial cells. Cells are either from allogneic, autologuous or xenogenic sources.

The term “chamber” refers to an element encapsulating and/or supporting cells, wherein cells are either suspended in a liquid medium or immobilized within an immobilizing biocompatible matrix wherein the liquid medium or the matrix sustains cell viability and function via the provision of the necessary nutrients and required factors. Typical suitable biocompatible matrix for immobilizing cells within the chamber of a device according to the invention comprises precipitated chitosan, alginate, collagen, synthetic polymers such as polyvinyl alcohol (PVA), microcarriers such as microspheres and the like, depending upon the cells to be encapsulated and their growing characteristics. According to an aspect of the invention, the cell-supporting biocompatible matrix is in a form of microbeads. According to another aspect of the invention, the cell-supporting biocompatible matrix is in a form of PVA matrix.

The term “semi-permeable membrane” refers to a biocompatible membrane which is permeable to said secreted antibody or fragment thereof, and impermeable to immune system materials from said host. Typically, a semi-permeable membrane is formed from a polymer including polyacrylates (including acrylic copolymers), polyvinylidenes, polyvinyl chloride copolymers, polyurethanes, polystyrenes, polyamides, cellulose acetates, cellulose nitrates, polysulfones (including polyethersulfones), polyphosphazenes, polyacrylonitriles, poly(acrylonitrile/covinyl chloride) and derivatives or combinations thereof. In one aspect, the semi-permeable membrane is capable of preventing cell-cell contact between cells inside the chamber and cells outside the device, thereby minimizing the deleterious effects of the host's immune system on the cells within its core upon implantation of the device into said host, such that the device functions for extended periods of time in vivo.

The term “biocompatible” means that, upon implantation in a host, it does not elicit a host response sufficient to detrimentally affect the device function or to render it inoperable. Such inoperability may occur, for example, by formation of a fibrotic structure around the membrane sufficient to limit diffusion of nutrients to the cells therein and/or limit the release of the secreted antibody or fragment thereof into the host. Detrimental effects may also include rejection of the device or release of toxic or pyrogenic compounds (e.g. synthetic polymer by-products) from the membrane to surrounding host tissue.

The term “device” includes any encapsulation device suitable for delivery of biomaterials such as proteins like antibodies or fragments thereof such as vascular shunts or “flow-through” systems, microcapsules or macrocapsules (e.g. hollow fibers or flat sheets) like described in Lysaght et al., 1999, Scientific American, 280(4), 76-82. Typically, the jacket can have any suitable configuration for maintaining cell viability and function and allowing the release of the antibody or fragment thereof. Suitable configurations include cylindrical, rectangular, disk-shaped, patch-shaped, ovoid, spherical or sheet-shaped. In a particular embodiment, a device according to the invention includes a biocompatible hollow fiber device (in Lysaght et al., 1999 above) wherein the chamber comprises PVA matrix. Typically, hollow fibers have an inside diameter/oustide of about 200 μm to about 1,000 μm; a wall thickness of about 10 μm to about 300 μm.

The term “antibody or fragment thereof” refers to any antibody or fragment thereof suitable as therapeutics. Examples of therapeutic antibodies or fragments thereof are provided in Hollinger et al., 2005, above & Devey et al., 2008, above. According to a particular embodiment, cells are expressing and secreting an antibody fragment (e.g. scFv-β1) such as described in Paganetti et al., 2005, J. Cell. Biol., 168(6), 863-8. In particular, the term “antibody or fragment thereof” includes single chain fragment variable (scFv) antibodies, fragment antigen binding (Fab) antibodies and immunoglobulin G (IgG).

The term “elements necessary for maintaining expressing cells alive” includes cell nutrients such as growth factors, vitamins, trace minerals, glucose, oxygen and the like.

The term “suffering from a disease or condition” means that a person is either presently subject to the signs and symptoms, or is more likely to develop such signs and symptoms than a normal person in the population.

The term “neurodegenerative disease or disorder” comprises a disease or a condition from the central nervous system (CNS) characterized by the progressive loss of structure or function of neurons, leading to their degeneration and to their death. It includes diseases or disorders such as Alzheimer's disease (AD), Parkinson's disease, Huntington's disease, amyotrophic lateral sclerosis (ALS) and muscular dystrophy. It further comprises neuro-inflammatory and/or demyelinating conditions or diseases comprising the degradation of the myelin around the axons. Demyelinating conditions or diseases comprise conditions wherein a process demyelinates cells such as multiple sclerosis, myelopathies, radiation induced demyelination, prion induced demyelinating condition or a spinal cord injury.

As used herein, “treatment” and “treating” and the like generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it such as a preventive early asymptomatic intervention; (b) inhibiting the disease, i.e., arresting its development; or relieving the disease, i.e., causing regression of the disease and/or its symptoms or conditions such as improvement or remediation of damage.

The term “effective amount” as used herein refers to an amount of at least one polypeptide or a pharmaceutical formulation thereof according to the invention that elicits the biological or medicinal response in a tissue, system, animal or human that is being sought. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented.

The term “efficacy” of a treatment according to the invention can be measured based on changes in the course of disease in response to a use or a method according to the invention. For example, the efficacy of a treatment for Alzheimer's disease may be assayed by behavioral and/or cognitive tests such as described in Mendez., 2006, International Journal of Psychiatry Medicine, 36(4), 401-412; Lalonde et al., 2002, Brain Research, 956(1), 35-44; Kelly et al., 2003, Neurobiology of Aging, 24(2), 365-378, or by neuroimaging using techniques such as positron emission tomograph (PET), single proton emission computed tomography (SPECT), Magnetic Resonance Imaging (MRI) and Magnetic Resonance Spectroscopy (MRS) described in Soares et al., 2008, Clinical Radiology, 64(1), 12-21; Scheltens, 2009, Dialogues in Clinical Neuroscience, 11(2), 191-9.

### Device According to the Invention

According to one embodiment, is provided a device for delivering an antibody or a fragment thereof to a host, comprising:

(a) A chamber comprising cells expressing and secreting said antibody or fragment thereof;

(b) A jacket encapsulating the chamber and providing a physical barrier between the said host and the chamber, the jacket having a permeability such that the secreted antibody or fragment thereof can diffuse therethrough into the host and the elements necessary for maintaining expressing cells alive can diffuse into the chamber.

According to a further embodiment, is provided a device according to the invention wherein the jacket is a semi-permeable membrane.

According to another further embodiment, is provided a device according to the invention wherein the semi-permeable membrane jacket has a molecular weight cutoff of between about 50 and 2000 kilodaltons (kDa).

According to another further embodiment, is provided a device according to the invention wherein the device is configured as a flat sheet.

According to another further embodiment, is provided a device according to the invention wherein the device is configured as a hollow fiber.

According to another further embodiment, is provided a device according to the invention wherein the cells are immobilized in a biocompatible matrix within the chamber and the cell-supporting biocompatible matrix is in the form of microbeads.

According to another further embodiment, is provided a device according to the invention wherein the cells are immobilized in a biocompatible matrix within the chamber and the cell-supporting biocompatible matrix is a PVA matrix.

According to another further embodiment, is provided a device according to the invention wherein the device is recoverable, i.e. may be retrieved from the host upon necessity.

According to another further embodiment, is provided a device according to the invention, wherein the device is refillable, in vitro or in vivo, with new secreting cells.

According to another further embodiment, is provided a device according to the invention wherein the chamber contains between about 1 and about 100×10⁶ cells. Typically, the number of cells encapsulated will depend on the device: e.g. about 1 cell to about 1,000 in the case of microcapsules, and 100 to about 100×10⁶ cells in the case of hollow fibers.

According to another further embodiment, is provided a device according to the invention wherein the chamber contains myoblasts as expressing and secreting cells.

According to another further embodiment, is provided a device according to the invention wherein the chamber contains fibroblasts as expressing and secreting cells.

According to another further embodiment, is provided a device according to the invention wherein the device is suitable to be implanted into a host.

According to another further embodiment, is provided a device according to the invention wherein the device is suitable to be implanted into the aqueous and vitreous humor of the eye from the host.

According to another further embodiment, is provided a device according to the invention wherein the device is suitable to be implanted into the central nervous system of the host.

According to another further embodiment, is provided a device according to the invention wherein the device is suitable to be implanted into the intrathecal space and/or the spinal cord from the host.

According to another further embodiment, is provided a device according to the invention wherein the device is suitable to be implanted subcutaneously into the host.

According to another further embodiment, is provided a device according to the invention wherein the device is suitable to be implanted intraperitoneally into the host.

According to another further embodiment, is provided a device according to the invention wherein the device is suitable to be implanted as a shunt in the blood system from the host.

According to another further embodiment, is provided a device according to the invention wherein the device is suitable to be implanted subepithelially into the host.

According to another further embodiment, is provided a device according to the invention wherein the antibody is a scFv.

According to another further embodiment, is provided a device according to the invention wherein the antibody is a Fab.

According to another further embodiment, is provided a device according to the invention wherein the antibody is an IgG.

According to another further embodiment, is provided a device according to the invention wherein the chamber contains cells expressing and secreting a scFv antibody targeted to the N-terminus of Amyloid beta (e.g. scFvβ1).

According to another further embodiment, is provided a device according to the invention wherein the chamber contains cells containing an expression regulatory element operatively linked to a nucleic acid sequence encoding said scFv antibody targeted to the N-terminus of Amyloid beta.

According to another further embodiment, is provided a device according to the invention wherein cells secretes a scFv antibody targeted to the N-terminus of Amyloid beta comprising the sequence set forth in SEQ ID NO: 1.

### Methods of the Invention

According to another aspect, the invention provides a method for delivering an antibody or a fragment thereof to a host, comprising implanting at least one device according to the invention into said host.

According to another aspect, the invention provides a method for treating a disease or disorder in a mammal comprising delivering an effective amount of an antibody or a fragment thereof by implantation in said mammal of at least one device according to the invention, said device producing a therapeutically effective amount of said antibody or a fragment thereof.

According to a further aspect, the invention provides a method according to the invention for the treatment of any disease treated by systemic antibody administration.

According to another aspect, the invention provides a method for treating nerve damage in a mammal comprising delivering an effective amount of an antibody or a fragment thereof by implantation in said mammal of at least one device according to the invention, said device producing a therapeutically effective amount of said antibody or a fragment thereof.

According to another aspect, the invention provides a method according to the invention wherein the host or the mammal is a patient suffering from a neurodegenerative disease or disorder.

According to another further aspect, the invention provides a method according to the invention wherein the host or the mammal is a patient suffering from Alzheimer's disease.

According to another further aspect, the invention provides a method according to the invention wherein the device is implanted in the brain.

According to another further aspect, the invention provides a method according to the invention wherein the device is implanted subcutaneously.

According to another further aspect, the invention provides a method according to the invention wherein the device is implanted intraperitonealy.

According to another further aspect, the invention provides a method according to the invention wherein the device is implanted subepithelially.

According to another aspect, the invention provides a use of a device according to the invention for the manufacture of a delivery system for delivering an antibody or a fragment thereof to a host.

According to one aspect, the uses, methods and devices of this invention are intended for use in a mammalian host, recipient, patient, subject or individual, preferably a primate, preferably a human.

According to another aspect, the uses, methods and devices of this invention are intended for the treatment of diseases or disorders that may be treated by antibody and/or antibody fragments.

According to one aspect, the methods, devices and delivery systems in the context of this invention are intended for use in a patient suffering from a disease or condition such a neurodegenerative disease or disorder, CNS disease or disorder (e.g. nerve damage, multiple sclerosis, Alzheimer's disease, Parkinson's disease), an eye disease or damage (e.g. age-related macular degeneration, retinitis pigmentosa), a cancer (e.g. tumors, neoplasms, leukemia), autoimmune disorders (e.g. Crohn's disease, arthritis, psoriasis), inflammatory responses (asthma, ulcerative colitis, transplant rejection), cardiovascular disorders (haemostasis, thrombosis) and infections (virus, bacteria).

The implantation sites for a device according to the invention include but are not limited to the central nervous system, including the brain (e.g. striatum, the cerebral cortex), spinal cord, cerebrospinal fluid, subarachnoid (intrathecal) space, lateral ventricles, aqueous and vitreous humor of the eye, sub-cutaneous space, intra-peritoneal space, intra-blood system and subepithelial space.

In a particular embodiment, the device according to the invention is surgically implanted in parietal cortex, such that the secreted antibodies or fragment thereof are delivered into the CNS without having to cross the blood brain barrier.

The device according to the invention may be retrieved from the host upon necessity (e.g. treatment end, replacement, and/or in case side-effects occur).

Methods and devices according to the invention present the particular advantage to minimize immunological responses by avoiding cell-to-cell contact between the host tissue and the implanted cells, while the device's membrane porosity allows the bi-directional diffusion of nutrients, oxygen and waste, and the outward diffusion of the scFv into the implanted tissue. The resulting therapeutic advantages thereof include the capacity to release antibodies or fragment thereof for a long time (e.g. at least 6-months) and the retrievability allowing either replacement or interruption of the treatment, offering a possible replacement therapy to the peripheral injections of antibodies.

In a particular embodiment, methods and devices according to the invention, wherein cells secretes antibody fragments such as those lacking the Fc-region present the additional advantage to further reduce both potential side effects occurring from T-cell immune mediated responses and treatment costs by providing continuous delivery of therapeutic antibodies for a long-term.

The following non-limiting embodiments are also provided:

1. An implantable device for delivering an antibody or a fragment thereof to a host, comprising:
(a) a chamber comprising cells expressing and secreting said antibody or fragment thereof; and
(b) a jacket encapsulating the chamber and providing a physical barrier between the said host and the chamber, the jacket having a permeability such that the secreted antibody or fragment thereof can diffuse therethrough into the host and the elements necessary for maintaining expressing cells alive can diffuse into the chamber;

2. A device according to embodiment 1, wherein the jacket is a semi-permeable membrane;

3. A device according to any of embodiments 1 to 2, wherein the device is configured as a flat sheet;

4. A device according to any of embodiments 1 to 2, wherein the device is configured as a hollow fiber;

5. A device according to any of embodiments 1 to 4, wherein the cells are immobilized in a biocompatible matrix within the chamber and the cell-supporting biocompatible matrix is in the form of microbeads;

6. A device according to any of embodiments 1 to 5, wherein the device is recoverable;

7. A device according to any embodiments 1 to 6, wherein the device is refillable, in vitro or in vivo, with new secreting cells;

8. A device according to any of embodiments 1 to 7, wherein the chamber contains myoblasts as expressing and secreting cells;

9. A device according to any of embodiments 1 to 7, wherein the chamber contains fibroblast as expressing and secreting cells;

10. A device according to any of embodiments 1 to 9, wherein the chamber contains cells expressing and secreting a scFv antibody targeted to the N-terminus of Amyloid beta;

11. A device according to any of embodiments 1 to 10, wherein the chamber contains cells expressing and secreting a scFv antibody targeted to the N-terminus of Amyloid beta comprising the sequence set forth in SEQ ID NO: 1;

12. Use of a device according to any one of embodiments 1 to 11 for the manufacture of a delivery system for delivering an antibody or a fragment thereof to a mammal;

13. A method for delivering an antibody or a fragment thereof to a host, comprising implanting at least one device according to any one of embodiments 1 to 11 into said host;

14. A method of treating a disease or disorder in a mammal comprising delivering an effective amount of an antibody or a fragment thereof by implantation in a mammal in need thereof of at least one device according to any one of embodiments 1 to 11, said device producing a therapeutically effective amount of said antibody or a fragment thereof;

15. A method according to any of embodiments 13 or 14, wherein the device is implanted in the brain;

16. A method according to any of embodiments 13 or 14, wherein the device is implanted subcutaneously;

17. A method according to any of embodiments 13 or 14, wherein the device is implanted intraperitonealy;

18. A method according to any of embodiments 13 or 14, wherein the device is implanted subepithelially;

19. A method according to any one of embodiments 13 to 18, wherein the host or mammal is a patient suffering from a neurodegenerative disease or disorder; and

20. A method according to embodiment 19 wherein the neurodegenerative disease or disorder is Alzheimer's disease.

References cited herein are hereby incorporated by reference in their entirety. The invention now being generally described, it will be more readily understood by reference to the following examples which are included merely for purposes of illustration of certain aspects and embodiments of the present invention, and are not intended to limit the invention in any way.

### EXAMPLES

The following abbreviations refer respectively to the definitions below:
AD (Alzheimer disease), APP (β-amyloid precursor protein), DMEM (Dulbecco's Modified Eagle Medium), EDTA (ethylenediaminetetraacetic acid), FBS (Foetal Bovine Serum), HBSS (Hanks Balanced Salt Solution), ID (Inner diameter), NDS (normal donkey serum), PBS (Phosphate Buffer Sulfate), PS (polysulfone), PVA (polyvinyl alcohol), OD (outer diameter), WT (wild-type).

### General Procedures & Conditions

Typically, a cell line is engineered for expressing and secreting a therapeutic antibody of interest or a fragment thereof by known methods such as described in Sambrook et al., 2001, Molecular cloning: a laboratory manual, 3^{rd} edition (Cold Spring Harbor Laboratory Press, New York, N.Y., USA), where for example a gene of interest is inserted into a suitable expression vector and the resulting expression vector containing the gene of interest may then be used to transfect the cell line to be used in the devices and methods of this invention. It will be appreciated that more than one gene may be inserted into a suitable expression vector. A wide variety of host/expression vector combinations may be used to express the gene encoding the desired antibody or antibody fragment according to the invention.

Typically, standard transfection techniques include electroporation, liposome-mediated, chemical methods (e.g. calcium phosphate), or physical methods (e.g. microinjection). Suitable promoters include, for example, the early and late promoters of SV40 (Simian virus 40), CMV (human cytomegalovirus), PGK (phosphoglycerate kinase) and any other promoters capable of controlling gene expression as described in Sambrook et al., 2001, above.

Suitable mammalian expression vectors include, for example plasmids such as pUC, pRK, pCDNA and the like.

Resulting transfected cells are then selected for their expression and secretion ability for the target antibody or fragment thereof by known methods such as described in Freshney, 2005, Culture of Animal Cells: A Manual of Basic Technique, 5^{th} edition (Wiley-liss, New York, N.Y., USA).

Selected cells are then loaded into the chamber of a device according to the invention. Typically, the cells are loaded using known methods such as described in Schneider et al., 2003, Molecular therapy, 7(4), 506-514; Schwenter et al., 2004, Human Gene Therapy 15(7), 669-680. Typically, cells loaded are kept in vitro in Dulbecco's modified eagle medium (DMEM), and fetal bovine serum (FBS) before implantation. The chamber is encapsulated into a jacket using known methods such as described in Schneider et al., 2003, above; Schwenter et al., 2004, above.

The device is then tested for cell secretion and permeability to the target antibody or fragment thereof by known methods such as described in Harlow and Lane 1998, Using Antibodies: A Laboratory manual [495] p. (Cold Spring Harbor Laboratory Press, NY, N.Y., USA) and implanted to the desired implantation site by known methods such as described in Aebischer et al., 1996, Nature medicine, 2(6), 696-699; Schneider et al., 2003, above; Sajadi et al., 2006, Neurobiology of disease 2006, 22(1), 119-129.

### Example 1

### Preparation of a Stable Cell Line Expressing SCFVβ1

In order to test the ability of a device according to the invention to deliver active antibodies in situ, a stable cell line expressing and secreting an antibody fragment of interest was engineered for encapsulation into a device of the invention.

### Construction of Expression Vectors

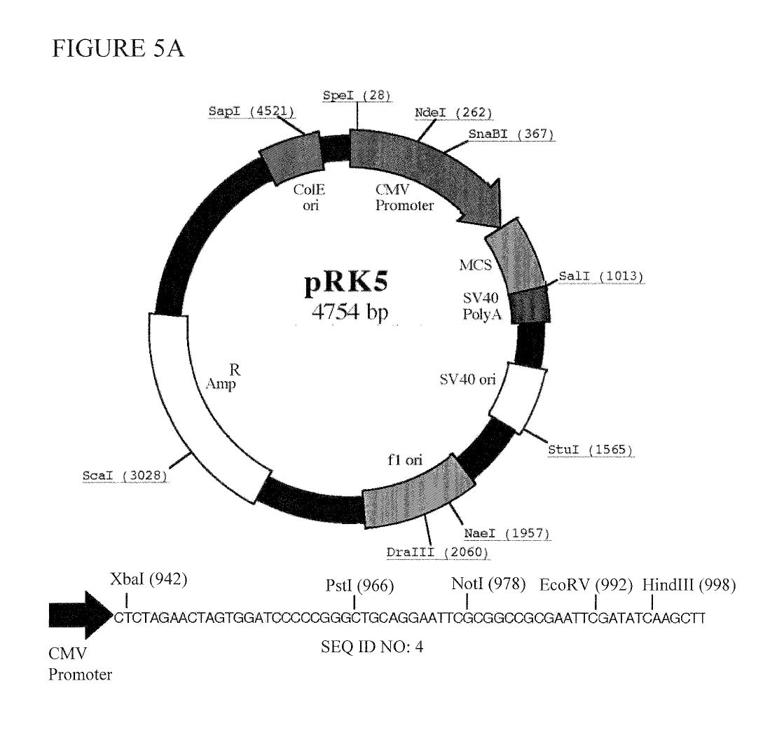

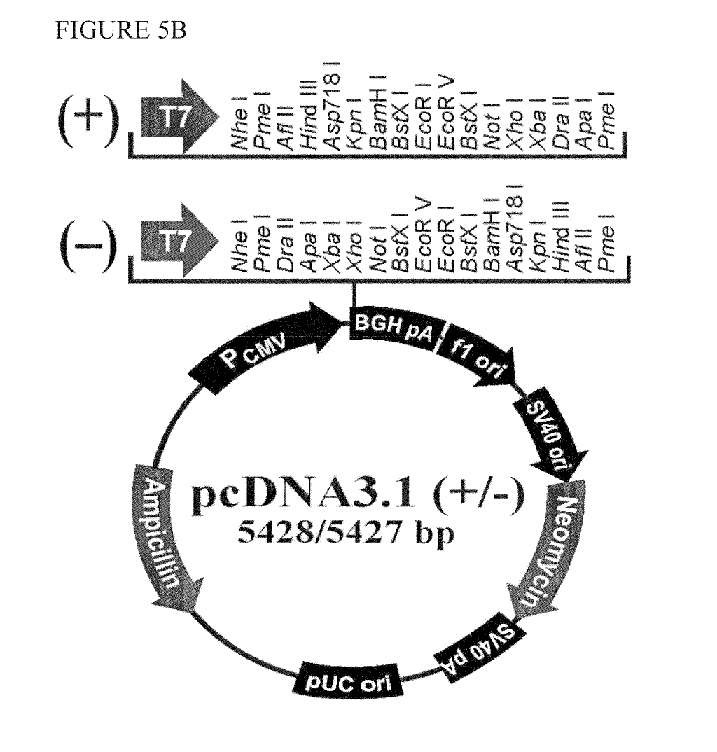

The monoclonal antibody β1 (Paganetti et al., 1996, J Neurosci. Res., 46(3), 283-293) which specifically binds to the EFRH tetrapeptide (SEQ ID NO: 1) adjacent to the β-secretase cleavage site of human APP. The β1 sequence was used as template for preparation of a pRK5-based expression plasmid (FIG. 5A, SEQ ID NO: 2) (BD PharMingen) encoding the single chain antibody named scFvβ1 or the full-length antibody named IgGβ1 (pRK5-scFvβ1 and pRK5-IgGβ1, respectively) or a pCDNA3.1-based expression plasmid (FIG. 5B, SEQ ID NO: 3) (Invitrogen) encoding the Fab fragment Fabβ1 (pCDNA3.1-Fabβ1).

The mature scFvβ1 consists of the light and heavy chain variable regions of β1 (132 and 120 residues, respectively) covalently linked by a GGGGS pentapeptide (SEQ ID NO: 5) repeated three times and displaying a His₆-tag at the C-terminus such as described in Paganetti et al., 2005, above.

The mature Fabβ1 consists of the β1 heavy chain depleted of CH2 and CH3 the two constant domains of the Fe region (260 residues) and the β1 light chain (247 residues). Fabβ1 is not tagged.

The mature IgGβ1 consists of the full length antibody with unaltered heavy and light chain (473 and 238 residues respectively) expressed in a single ORF and linked by the self cleaving viral 2A peptide (Fang et al., 2005, Nature Biotechnology 23(5), 584-590).

Cell Transfection & Obtention of Genetically Modified C2C12Cells for scFvβ1 Secretion

C2C12 mouse myoblasts at 80% confluency were co-transfected using Lipofectamine 2000 (Invitrogen) with 3 μg of a ScaI-linearized pRK5-scFvβ1 and with 3 μg SeaI-linearized pcDNA3.1 to confer geneticin resistance and allow selection of stable clones. Single clones were screened for transgene secretion by immunoprecipitation of ectopically expressed scFvβ1 with mouse monoclonal anti-His antibody (MCA1396, Serotec). As control cells transfected with the pRK5 plasmid without the transgene (mock) were used.

Amongst the different clones obtained after transfection, one positive stable clone that maintained high level of expression of scFvβ1 (cells were secreting 31.1±0.6 ng of scFvβ1 per 50,000 cells per 24 hours (n=4)) for several months in culture was selected for its subsequent encapsulation into hollow fiber membranes.

### Preparation and Selection of Cell Clones Expressing Fabβ1

C2C12 mouse myoblasts at 80% confluency were transfected using Lipofectamine 2000 (Invitrogen) with 3 μg of a SeaI-linearized pCDNA3.1-Fabβ1 and selected with geneticin. Single clones were screened for transgene secretion by immunoprecipitation of ectopically expressed Fabβ1 with anti-Fab antibody (M6898, SIGMA).

### Preparation and Selection of Cell Clones Expressing IgGβ1

C2C12 mouse myoblasts at 80% confluency were co-transfected using Lipofectamine 2000 (Invitrogen) with 3 μg of a SeaI-linearized pRK5-IgGβ1 and ScaI-linearized pcDNA3 (3:1 ratio) and selected with geneticin. Single clones were screened for transgene secretion by isolation of ectopically expressed IgGβ1 with ProteinA beads (P4931, SIGMA).

### Example 2

### Preparation of the Device to be Implanted (Hollow Fiber Capsule)

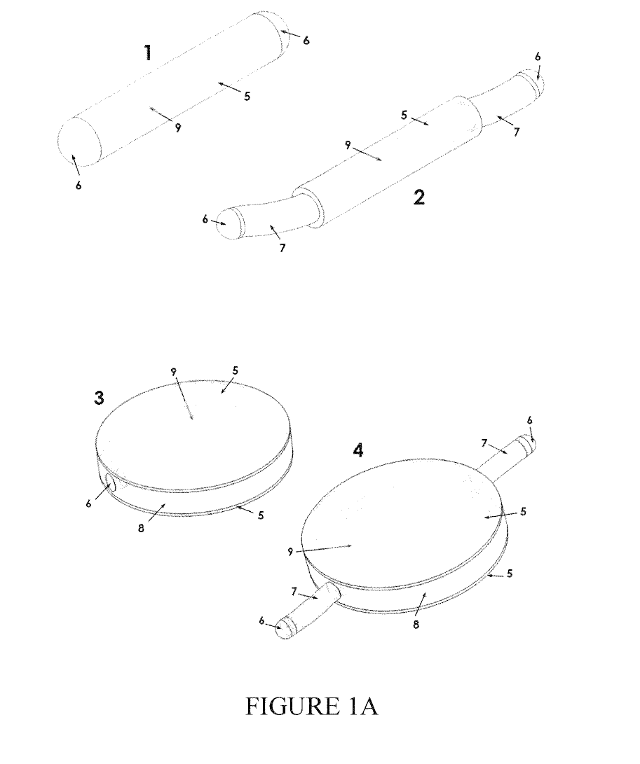

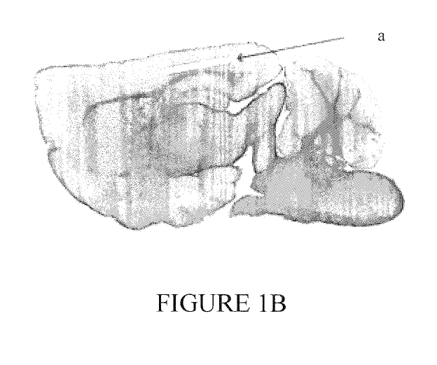

A device according to the invention was prepared in the form of a hollow fiber capsule wherein the chamber comprises a PVA matrix and the jacket comprises a PS membrane, for subsequent loading with the C2C12cells selected above, as follow (FIG. 1A, (1)): stainless steel tips (EFD, http://www.efd-inc.com/Tips/) were detached from their hub and connected to the tip of 4 mm long PS hollow fiber semi-permeable membranes (Minntec) of a molecular weight cutoff: ˜100 kDa, allowing the diffusion of the ˜27 kDa scFvβ1 fragments using a photo-polymerized acrylic-based glue (Ablestic Laboratories) to facilitate the loading of cells. The plastic hub serves for introducing the Hamilton syringe containing the cells into the capsule. Hollow fiber membranes were filled with a polyvinyl alcohol sponge (Rippey Corporation) used for cell anchorage, and were obtained using a hollow drill with an internal diameter corresponding to the inner dimensions of the capsule. The PVA rods were sonicated in ultra pure water and dried. The matrices were inserted into the 4 mm long semi-permeable PS hollow fibers and sealed. Capsules were sterilized with ethylene oxide and kept 10 days at room temperature to eliminate traces of gas.

Scanning electron microscopy was performed to assess the homogeneous surface structure and porosity of the hollow fiber membrane using a Philips XLF30 field emission gun scanning electron microscope (FEG SEM) at x10,000, equipped with an Everhart-Thornley secondary-electron (SE). Membranes were dehydrated in alcohol baths from 70 to 100% followed by a 400 Å gold plasma coating for visualization at different resolutions.

### Example 3

### Encapsulation of the Cells into the Device

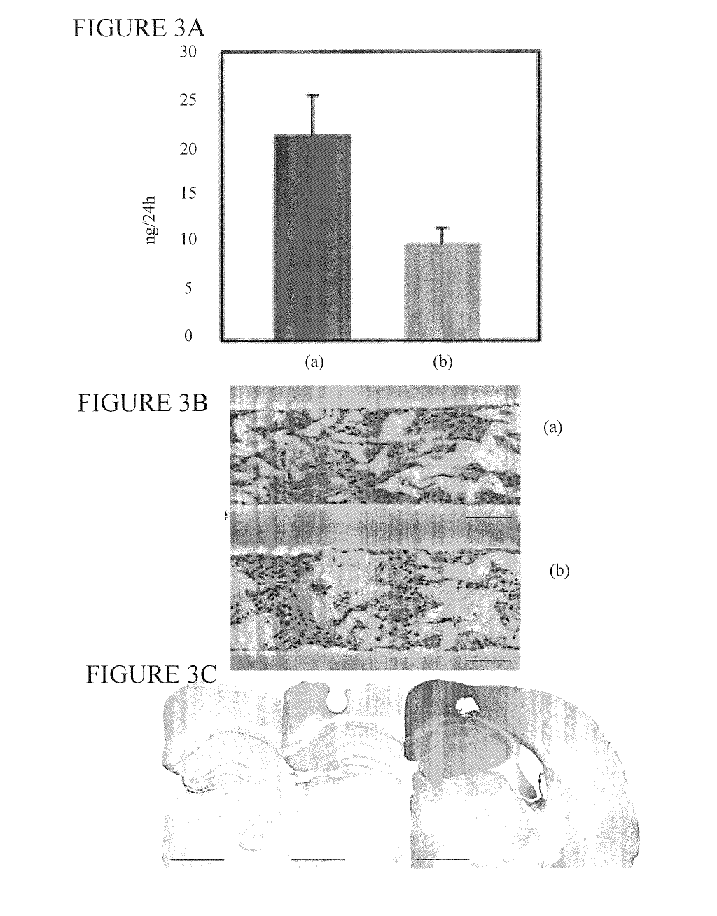

The cells prepared under Example 1 were loaded into the device designed under Example 2 as follows: C2C12-scFvβ1 clones were harvested using 0.125% trypsin-EDTA and diluted with 50% DMEM 5% FBS+50% HBSS to achieve a suspension of 50,000 cells/μl culture medium. Using a 50-μl syringe (Hamilton) fitted with an adaptor hub, 1 μl of cell suspension was injected into the capsule. The hubs and steel tip were removed and the extremity of the capsules sealed. The capsules were washed in HBSS 1% FBS for 1 hour and then transferred to DMEM, 10% FBS (5% CO₂, 37° C.) for 21 days before implantation into the brain cortex of mice. Once encapsulated and kept in vitro for 3 months, cells still produced 17 ng/24 hrs of scFvβ1. Cell survival within the device measured in vitro as well as in vivo at 2 and 6 months post-implantation, showed evenly distributed C2C12 cells intermingled with the polyvinyl alcohol (PVA) matrix (FIG. 3A).

### Example 4

### Implantation of the Device and Therapeutic Effect

A device according to the invention prepared as described in the above Examples was implanted bilaterally into the posterio-parietal cortex of APP23 transgenic mice (FIG. 1B), a mice model for AD-like pathology described in Sturchler et al., 1997, Proc. Natl. Acad. Sci. USA., 94, 13287-92 where mice express the human APP with the Swedish mutation in seven-fold excess compared with the endogeneous murine APP, as follows: eight-month age matched female APP23 mice were housed in 12 h light/dark cycle, with ad libitum access to food and water. Deeply anesthetized animals were placed into the stereotaxic frame (Kopf Instruments) equipped with a precise micromanipulator with a horizontal arm in a 79° angle. Four-millimeter long hollow fiber capsules were bilaterally implanted in the cortex (anterior-posterior: −1.1 mm, lateral: ±1.2 mm, ventral: −5.5 mm, tooth bar: −7 mm, degree of arm 79°, according to the atlas of Paxinos and Franklin, 2004, The mouse brain in stereotaxic coordinates, [264] (Academic, San Diego, Calif., 2004)) of 16 female APP23 mice.

The first group consisted in 9 APP23 mice implanted with C2C12-mock, and the second with 7 APP23 mice implanted with C2C12-scFvβ1 hollow fiber capsules. All animal experiments were approved by the local veterinary office and carried out in accordance with the European Community Council Directive (86/609/EEC) for care and use of laboratory animals.

Localization of the implanted hollow fiber capsules in the APP23 brain cortex of APP23-scFvβ1 (n=5) and APP23-mock (n=5) mice was visualized from coronal in vivo images using an MRI system as follows: APP23-mock, and APP23-scFvβ1 mice were anesthetized using 1.3±0.2% of isoflurane in oxygen using a nose mask. Body temperature was kept at 37±0.5° C. Images were acquired on an MRI System (Varian) interfaced to a 14.1 Tesla magnet with a 26-cm horizontal bore (Magnex Scientific). A home-built quadrature surface coil consisting of two geometrically decoupled 14-mm-diameter single loops was used as a transceiver. Localizer images were obtained in the coronal plane using a multislice fast spin echo protocol with an echo time of 60 ms, repetition times of 5,000 ms, a slice thickness of 0.6 mm and an isotropic in-plane resolution of 78 μm.

### Behavioral Analysis

Behavioral testing was performed in female APP23 mice (n=21) and their WT-littermates (n=7) during the light cycle period (8 am to 2 pm). In all tests, mice trajectories were recorded with a vertically mounted camera and analyzed with a video tracking software (Ethovision 3.1.16, Noldus). In order to maximize homogeneity of groups before scFvβ1 capsule implantation, 7 month-old APP23 mice (n=21) were tested for anxiety-like, locomotor and exploration behaviors in the elevated plus maze (EPM), the open field and the novel object (OF/NO) reactivity test and subsequently matched so that not significant differences were observed between the subgroups (APP23 n=5, APP23-mock n=9, APP23-scFvβ1 n=7).

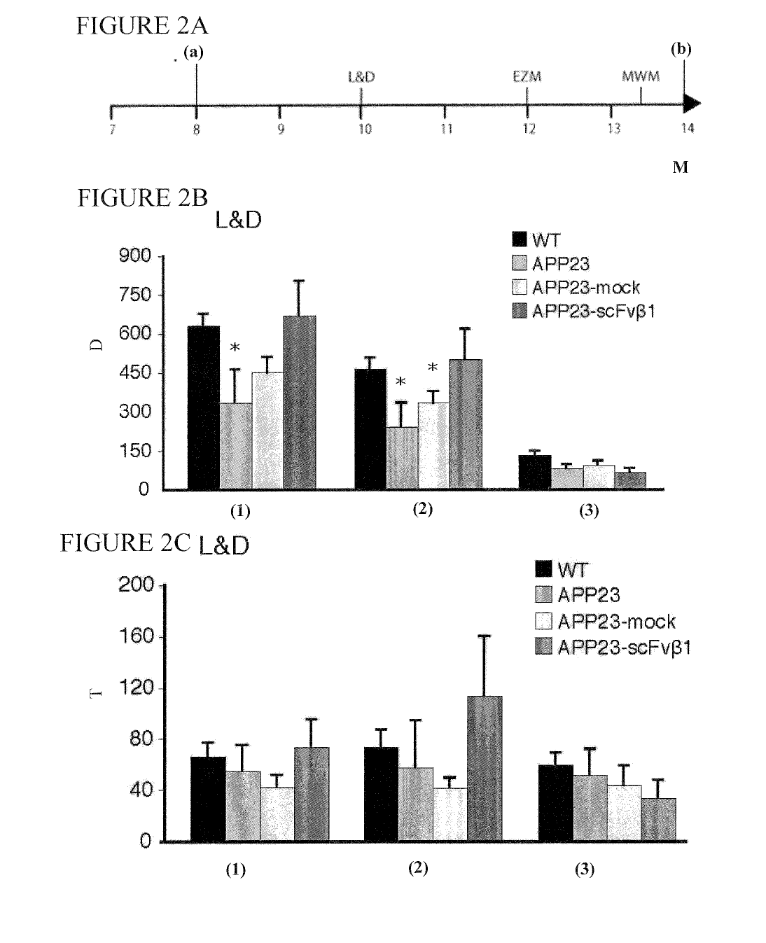

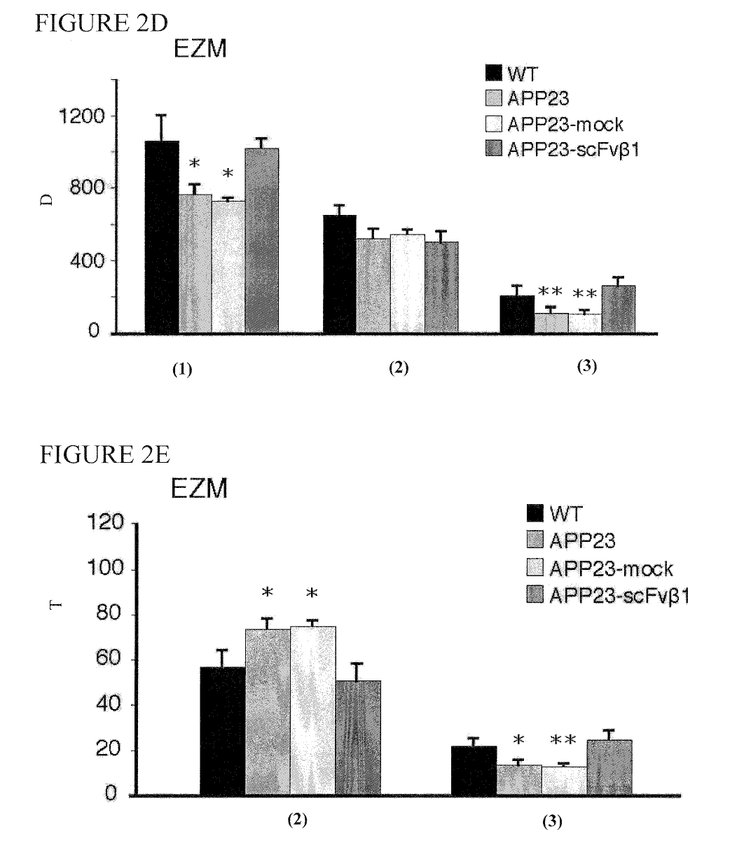

Then, the behavioral impact of the scFvβ1 treatment animals was evaluated in two different anxiety-like tests, light and dark (L&D) and elevated zero maze (EZM) and in the Morris water maze (MWM) for cognitive functions according to the experiement protocol on FIG. 2A. Pre-implantation and post-implantation behavioral data were analyzed using a one-way analysis of variance (ANOVA) followed by an LSD post-hoc test, where appropriate. Water maze data were analyzed using ANOVA for repeated measures for general performance across spatial learning in trials; one-way ANOVAs were applied on block data for each testing day followed by a post-hoc LSD test. Significance of results was accepted at p≦0.05. Data are expressed as means±S.E.M.

In the L&D test, anxiety-like behavior was indicated by the distance traveled and time spent in the open lighted and anxiogenic arena (FIGS. 2B & C). Significant differences were observed between the APP23-scFvβ1 and WT-littermates groups in the total distance traveled in the light quadrant compared to the APP23 group (p<0.05). Similarly, APP23-scFvβ1 animals and both APP23 and APP23-mock groups showed a significant difference in the distance covered in the light quadrant (without the transition zone) (p<0.05). No differences between groups were found in distance moved and time spent in the transition zone, indicating a lack of changes in general exploratory behavior.

In the EZM test, APP23 and APP23-mock mice moved less in the maze than WT mice, due to a specific reduction in both total movement in the arena (p<0.05), and in the open arms (p<0.01) (FIGS. 3D & E). Differences were also observed in the time spent in the open arms (p<0.05) and close arms (p<0.05) indicating enhanced anxiety-like behavior in the non-scFvβ1 treated APP23 mutants. APP23-scFvβ1 mice differed from the untreated and mock mutants, but not from WT controls in their total movement in the arena (p<0.05), in the open arms (p<0.001) and for the time spent in the open (p<0.01) and closed arms (p<0.01), indicating reduced anxiety-like behavior.

The impact of scFvβ1 treatment on cognitive functions was also evaluated 5.5 months after treatment on spatial learning and working memory functions in the MWM test. No significant differences were found between WT-littermates and all APP23 groups in their distance moved to find the hidden platform over the three spatial learning sessions (days 1-3), and did not differ from each other in their daily average distance to reach the platform. Following the reversal learning (day 4), the APP23-scFvβ1 and WT-littermates showed a trend (p<0.1) to reach the platform in shorter distance compared with both control APP23 groups. No locomotor or visual deficits were observed between groups on the visual platform task (day 8).

Animals were then challenged to delayed-matching-to-place (DMTP) trials such as described in Steel et al., 1999, Hippocampus, 9, 118-36 (days 11 and 12). On the second day of the DMTP trials, APP23 and APP23-mock mice showed impaired performance with regards to both WT (p<0.05) and APP23-scFvβ1 (p<0.01) mice. Therefore, scFvβ1 treatment improved mice working memory to re-locate the platform in the novel position as indicated by their shorter average escape distances. An analysis of a compound measure of mice's behavior by collapsing data corresponding to the second trials for each of the cognitive challenges given in the MWM (i.e., days 1, 4, 11 and 12) indicated that WT and APP23-scFvβ1 mice performed better in their learning strategy than the APP23 and APP23-mock mice (all p<0.05).

Behavioral evaluation during the course of the treatment according to the invention showed that reduction of Aβ levels after scFvβ1 delivery modified behavioral traits related to anxiety and working memory in the APP23 mice. Thus, APP23-scFvβ1 mice showed reduced anxiety-like behavior during both the L&D and the EZM tests, at two- and four-months post-scFvβ1 treatment respectively; and showed improved learning strategies during the second MWM learning trials, and displayed improved working memory in the DMTP paradigm.

### Analysis of Brain Samples and Retrieved Capsules

Mice were sacrificed and the capsules were retrieved six months post-implantation for evaluation of scFvβ1 secretion. Mice were deeply anesthetized by an overdose of pentobarbital and transcardially perfused with ice-cold PBS. The brain was recovered and capsules were removed and placed in DMEM 10% FBS at 37° C., 5% CO₂.

Brains were sagittally sectioned in two; the hippocampus and cortex of the left hemisphere were immediately dissected for protein extraction. The right hemisphere was immediately fixed in 4% paraformaldehyde (Fluka-Sigma) for 2 hours and then transferred into 25% sucrose in PBS and placed at 4° C. overnight. Twenty-five μm thick coronal sections were harvested on a freezing stage sliding microtome (Leica SM2400). Entire brain slices were captured in the bright field with a motorized stage on the Leica DM5500 microscope (software: Leica LAS) at a 10× resolution. Each brain slice was segmented from the background to obtain the brain surface followed by the quantification of the size of the amyloid plaques. Both processes were performed through different channel manipulation of the red, green and blue (RGB) images and then by object detection. Artifacts were avoided by filtration on shape and size. The semi-automated journals were performed with METAMORPH 7.5 (Universal-Imaging). Cerebral amyloid angiopathy and microhemorrhages were quantified using a double staining with 4G8 antibody with 3,3′-diaminobenzidine solution (DAB) as described below and counterstained with the Prussian blue method as described below for hemosiderin-positive microglial cells in eight coronal brain sections (150 μm apart) throughout the sector where the capsule were implanted. DAB protocol: Free-floating sections were washed three times with PBS, then quenched with 0.1% phenyl hydrazine (Merck, Whitehouse Station, N.J., USA) in PBS at 37° C. for 1 hour, and incubated for 2 hours at room temperature in a blocking solution of 10% NDS, 1% Albumin from Bovine Serum and 0.5% triton X-100 (Sigma, St. Louis, Mo., USA) in PBS. Overnight incubation with anti-histidine tag monoclonal antibody in blocking buffer at 4° C. was followed by a-2-hour incubation with biotinylated goat anti-mouse immunoglobulins (Vector Laboratories, Burlingame, Calif., USA). Slices were subsequently incubated in avidin-biotin-peroxidase solution (Vector Laboratories, Burlingame, Calif., USA) for 30 minutes. Slices were finally revealed with 3,3′-diaminobenzidine solution (Pierce, Rockford, Ill., USA) and mounted on glass slides. Prussian blue protocol: Free-floating sections we washed three times with PBS, and transferred to 10% hydrochloric acid mixed with 10% of Potassium Ferrocyanide solution. Sections were washed 3 times in distilled water, and mounted on glass slides.

Retrieved capsules were fixed overnight in 10% formalin and 1% picric acid and dehydrated under an alcohol cycle in preparation for glycol-methacrylate embedding (Leica Instruments). The capsules were cut at 9 μm-thickness using a LEICA microtome equipped with glass knifes and stained with hematoxylin-eosin (HE).

The above histological analysis of the retrieved capsules confirmed the survival of the cells 6 months following implantation and the sustained release of scFvβ1 fragments throughout the implantation period (release of 10±1.5 ng of scFvβ1/24 h compared to the 21±4 ng of scFvβ1/24 h at the time of implantation) (FIGS. 3A & B).

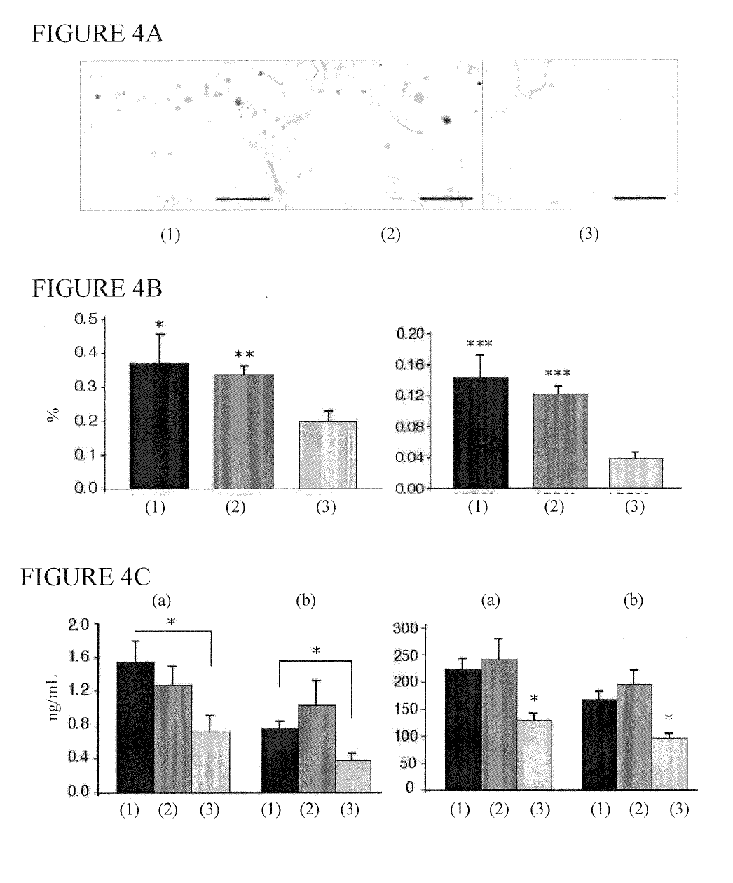

The total Aβ load was ascertained six months post-scFvβ1 treatment using immunohistochemical and congophilic staining as described below. In 14-month-old APP23 mice, Aβ plaque deposition was regionally distributed throughout the olfactory bulb, the cortex (although more densely concentrated in the ‘parietal’ and ‘occipital’ regions), and, to a lesser extent in the hippocampus. Congo red staining revealed that the size of Aβ insoluble plaques (FIG. 4A) was significantly reduced in the brain slices of APP23-scFvβ1 animals with regards to both APP23 (p<0.05) and APP23-mock (p<0.01) with a marked clearance in the hippocampus and posterio-parietal cortex regions as compared to APP23 and APP23-mock (all p<0.001) (FIG. 4B). ELISA from brain homogenates revealed that the soluble Aβ levels were significantly lower in APP23-scFvβ1 in the posterio-parietal cortex and hippocampus as compared to APP23 (p<0.05) (FIG. 4C left), and a trend was observed with APP23-mock (p<0.1). The insoluble levels of Aβ were significantly reduced in scFvβ1 antibody treated animals in the posterio-parietal cortex and hippocampus (all p<0.05) (FIG. 4C right), confirming the observations from the congophilic quantification.

Anti-histidine tag immunohistochemical detection of scFvβ1 fragments was performed in paraformaldehyde-fixed brain sections through the use of an anti-histidine tag mouse monoclonal antibody (Serotec), followed by peroxidase treatment using the M.O.M. immunodetection kit (Vector Laboratories), and revealed with the 3,3′-diaminobenzidine (DAB) (Pierce). It showed the presence of scFvβ1 surrounding the implantation site, covering the cortex (posterio-parietal region), the hippocampus (dorsal region at the CA1, CA2, CA3, CA4, and dentate gyrus) (FIG. 3C), and, to a lesser extent, other areas near the implanted capsules. No scFvβ1 was detected in the APP23 and APP23-mock brains.

Amyloid beta was detected using immunohistochemical detection with antibody anti-amyloid beta 4G8 (Acris) (Dudal et al., 2004, Neurobiology of aging 25(7), 861-871), and with the congo red histology (Wilcock et al., 2006, Nature Protocols, 1, 1591-5) on ten coronal brain sections (100 μm apart; every 4^{th} sections) taken from each animal in the region where the capsule was placed.

Histological and biochemical markers of inflammation revealed that scFvβ1-brain implants did not elicit an immune/tissue reaction, as determined by the absence of hemorrhages (Prussian blue method as described above), perivascular cuffings (cresyl violet as described below), and microglia markers mouse anti-glial fibrillary acidic protein (Novus biologicals, USA), and rabbit anti-Ibal (Wako, Japan) (Towne et al., 2008, Molecular Therapy, 16(6), 1018-1025). Cresyl violet staining: Slides were hydrated to distilled water, then placed slides in cresyl violet acetate solution for 5 minutes and rinsed in three changes of distilled water and dehydrated in graded alcohols. Slides were then cleared in three or four changes of xylene and mounted in glass slides.

We claim:

1. An implantable device for delivering an antibody or a fragment thereof to a host, comprising:
(a) a chamber comprising cells expressing and secreting said antibody or fragment thereof; and
(b) a jacket encapsulating the chamber and providing a physical barrier between the said host and the chamber, the jacket having a permeability such that the secreted antibody or fragment thereof can diffuse therethrough into the host and the elements necessary for maintaining expressing cells alive can diffuse into the chamber.

2. The device according to claim 1, wherein the jacket is a semi-permeable membrane.

3. The device according to claim 1, wherein the device is configured as a flat sheet.

4. The device according to claim 1, wherein the device is configured as a hollow fiber.

5. The device according to claim 1, wherein the cells are immobilized in a biocompatible matrix within the chamber and the cell-supporting biocompatible matrix is in the form of microbeads.

6. The device according to claim 1, wherein the device is recoverable.

7. The device according to claim 1, wherein the device is refillable, in vitro or in vivo, with new secreting cells.

8. The device according to claim 1, wherein the chamber contains myoblasts as expressing and secreting cells.

9. The device according to claim 1, wherein the chamber contains fibroblast as expressing and secreting cells.

10. The device according to claim 1, wherein the chamber contains cells expressing and secreting a scFv antibody targeted to the N-terminus of Amyloid beta.

11. The device according to claim 1, wherein the chamber contains cells expressing and secreting a scFv antibody targeted to the N-terminus of Amyloid beta comprising the sequence set forth in SEQ ID NO: 1.

12. A method for delivering an antibody or a fragment thereof to a host, comprising implanting at least one device according to claim 1 into said host.

13. A method of treating a disease or disorder in a mammal comprising delivering an effective amount of an antibody or a fragment thereof by implantation in a mammal in need thereof of at least one device according to claim 1, said device producing a therapeutically effective amount of said antibody or a fragment thereof.

14. The method according to claim 13, wherein the device is implanted in the brain.

15. The method according to claim 13, wherein the device is implanted subcutaneously.

16. The method according to claim 13, wherein the device is implanted intraperitonealy.

17. The method according to claim 13, wherein the device is implanted subepithelially.

18. The method according to claim 13, wherein the host or mammal is a patient suffering from a neurodegenerative disease or disorder.

19. The method according to claim 18, wherein the neurodegenerative disease or disorder is Alzheimer's disease.
